(19) United States
(12) Patent Application Publication
(10) Pub. No.: US 2020/0211705 A1
(43) Pub. Date: Jul. 2, 2020

(54) CONDENSED SURGIAL TREATMENT PROCESS FOR BARIATRIC PATIENTS

(71) Applicant: Thomas Umbach, Las Vegas, NV (US)

(72) Inventor: Thomas Umbach, Las Vegas, NV (US)

(21) Appl. No.: 16/238,014

(22) Filed: Jan. 2, 2019

(51) Int. Cl. G16H 50/20; G16H 10/20; G16H 40/20

(52) U.S. Cl. CPC G16H 50/20; G16H 40/20; G16H 10/20

(57) ABSTRACT

A condensed surgical treatment process for bariatric patients includes a qualification process whereby patient indicators are used to qualify a patient for bariatric surgery. Once qualified, scheduling of consultations and tests, patient evaluations, and the bariatric surgery itself within a condensed timeframe can occur. Steps of the condensed surgical treatment process occur within a predefined division of time to ensure the condensed surgical treatment process is completed within a condensed time period.

### BACKGROUND OF THE INVENTION

### 1. Field of the Invention

The invention relates to bariatric surgery and in particular to condensed surgical treatments for bariatric patients.

### 2. Related Art

Bariatric surgery is typically a difficult process that takes place across an extended timeline. Typically, patients seeking bariatric surgery must first meet particular requirements, such as a particular BMI, and have any medical ailments verified. In addition, lengthy supervised medical weight loss visits without weight gain can be required. In many cases, nine months of supervised medical weight loss visits without weight gain can be required.

After the requirements are met, the patient can then schedule tests for their surgery. However, the tests and their follow-ups are themselves a long-term process that typically requires a patient to visit a number of testing clinics or laboratories.

From the discussion that follows, it will become apparent that the present invention addresses the deficiencies associated with the prior art while providing numerous additional advantages and benefits not contemplated or possible with prior art constructions.

### SUMMARY OF THE INVENTION

A condensed surgical treatment process for bariatric patients is disclosed herein. As will be described further herein, the condensed surgical treatment process is advantageous in that it provides patients with a shortened and predictable time to obtain treatment. In this manner, complications or health issues relating to the patient's weight can be addressed more rapidly or avoided.

In addition, the condensed surgical treatment process utilizes a qualification process based on patient indicators rather than traditional processes which are significantly more time consuming and disruptive to a patient's daily life. Moreover, the condensed surgical treatment process is scheduled with providers that have a turnaround time within a selected threshold to permit the overall condensed surgical treatment process to occur in a shortened timeframe.

Various embodiments of a condensed surgical treatment process are disclosed herein. To illustrate, in one exemplary embodiment, a method for treating bariatric patients with bariatric surgery in a condensed time period is disclosed, with such method comprising collecting a plurality of patient indicators from a patient via a collection interface, determining if the patient qualifies for bariatric surgery in a condensed time period using the patient indicators, and if the patient is qualified for bariatric surgery in a condensed time periods, scheduling and providing testing, patient evaluation, and bariatric surgery to the patient, each within a predefined division of time and without any intervening period of time therebetween.

The patient indicators may include whether the patient has had weight loss surgery before, whether the patient has a weight or BMI beyond a particular threshold, whether the patient can walk 200 feet without chest pain or shortness of breath, whether the patient is a non-smoker, and whether the patient has commercial insurance coverage.

The patient may be qualified for condensed surgical treatment if each of the plurality of patient indicators is affirmed. It is noted that, in some embodiments, the patient qualifies for condensed surgical treatment if one or more predefined subsets of the plurality of patient indicators is affirmed.

In another exemplary embodiment, a method for condensed surgical treatment of bariatric patients is disclosed, with such method comprising collecting a plurality of patient indicators from a patient via a collection interface, determining if the patient qualifies for condensed surgical treatment using the patient indicators, and if the patient is qualified for condensed surgical treatment, scheduling and providing testing, patient evaluation, and bariatric surgery to the patient, each within a predefined division of time.

The patient indicators may include whether the patient has had weight loss surgery before, whether the patient has a weight or BMI beyond a particular threshold, whether the patient can walk 200 feet without chest pain or shortness of breath, whether the patient is a non-smoker, and whether the patient has commercial insurance coverage.

The patient may be qualified for condensed surgical treatment if each of the plurality of patient indicators is affirmed. Alternatively, in some embodiments, a patient may be qualified for condensed surgical treatment if one or more predefined subsets of the plurality of patient indicators is affirmed.

A post-operative procedure may be provided after the bariatric surgery. In addition, travel arrangements may be scheduled for the patient if the patient is qualified.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 is a flow diagram illustrating an exemplary condensed surgical treatment process for bariatric patients;

FIG. 2 is a flow diagram illustrating an exemplary multi-track condensed surgical treatment process for bariatric patients;

FIG. 3 is a flow diagram illustrating an exemplary condensed surgical treatment process for bariatric patients;

FIG. 4 is a block diagram illustrating an exemplary qualification system and components thereof; and

FIG. 5 is a flow diagram illustrating a qualification process using collected patient indicators.

### DETAILED DESCRIPTION OF THE INVENTION

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

The condensed surgical treatment process for bariatric patients disclosed herein reduces or eliminates many of the difficulties associated with traditional bariatric surgery. In addition, the condensed surgical treatment process greatly reduces the timeframe required for bariatric surgery.

The shortened timeframe is advantageous for practical and health reasons. Bariatric patients typically have health issues relating to their weight. As such, the condensed surgical treatment process can treat these issues more rapidly and help bariatric patients address or avoid, among other related health issues, heart disease, high blood pressure, and diabetes as well as their weight. As a matter of practicality, the shortened timeframe of the condensed surgical treatment process reduces interruption and disruption to a patient's daily life.

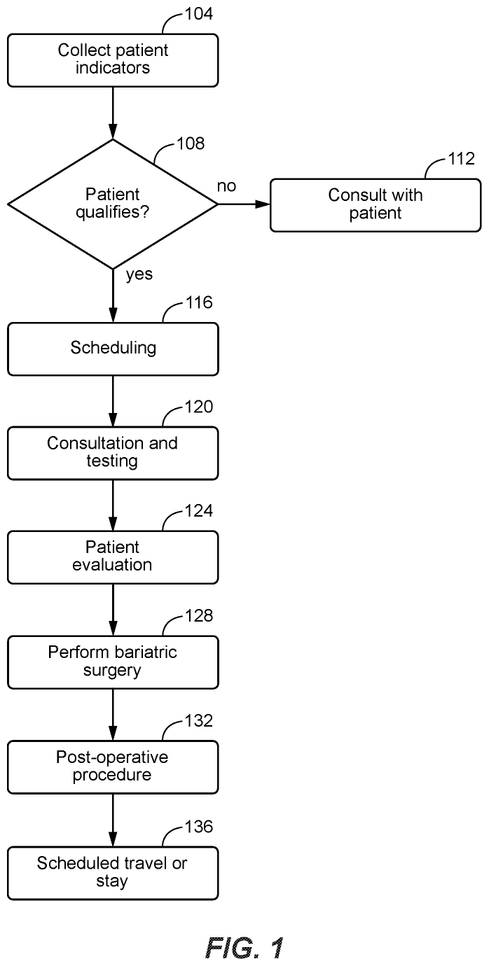

FIG. 1 is a flow diagram illustrating an exemplary condensed surgical treatment process. At a step 104, one or more patient indicators may be collected from a patient. In general, one or more patient indicators will be used to qualify the patient for bariatric surgery. Since the qualification or disqualification for a condensed surgical treatment process may be based on patient indicators, the time period for the condensed surgical treatment process is significantly shortened, especially relative to the timeframe to undergo traditional bariatric surgery.

At a decision step 108, it is determined whether the patient qualifies or is “cleared” for surgery. The qualification is based on the patient indicators collected previously. As will be described further below, patient indicators will typically be characteristics of the patient's physical and sometimes mental state. The patient indicators may be used to determine the risk to the patient from bariatric surgery and ultimately qualify or disqualify the patient for a condensed surgical treatment process.

If the risk is not within acceptable tolerances the patient may be disqualified at decision step 108. A patient consultation may be provided at a step 112 for disqualified patients. For instance, a physician may discuss the disqualification, other bariatric treatments, or both with the patient. In addition, such patient may still be a potential candidate for a traditional lengthier bariatric surgery process.

If the risk is within acceptable tolerances the patient may be qualified at decision step 108. At a step 116, scheduling may then occur. In one or more embodiments, scheduling includes scheduling of tests and evaluations as well as the bariatric surgery itself. This is unlike traditional emergency or operating room surgeries where testing is not scheduled for the patient. In addition, as will be described further below, the steps of a condensed surgical treatment process may be scheduled within predefined divisions of time to help ensure an overall shortened or condensed time period for the condensed surgical treatment process. Scheduling may optionally also include scheduling of post-operative procedures, travel arrangements, or both.

As shown in the embodiment of FIG. 1, consultation and testing at a step 120, patient evaluation at a step 124, bariatric surgery at a step 128, post-operative procedure at a step 132, and travel or a stay at a step 136 may be scheduled as part of the scheduling process at step 116. It is noted that, in some embodiments, scheduling need not include scheduling of post-operative procedures or travel or stay arrangements and these steps 132, 136 may take place in an ordinary course of time or as desired in such embodiments.

As alluded to above, scheduling will typically include scheduling for both the patient and providers. In one or more embodiments, scheduling will be limited to onsite or in-house providers to help further shorten the time period for the condensed surgical treatment process. Alternatively, or in addition, testing and evaluation or other steps may be scheduled with providers having a 24-hour turnaround or less for the same reason.

At a step 120, consultation and testing may occur. Some exemplary tests include lab work and a stress echocardiogram. Additional or other tests may be performed as well, such as based on collected patient indicators. It will be understood that consultation and testing may indicate that a patient has a higher risk than is acceptable. In such case, the condensed surgical treatment process will accordingly be terminated.

A consultation may include a discussion regarding the condensed surgical treatment process, risks thereof, patient questions, expectations, and the like. It is contemplated that consultation may occur in connection with other steps of a condensed surgical treatment process as well. A consultation may be provided by the patient's condensed surgical treatment process coordinator, surgeon, dietitian or other provider.

At a step 124, a patient evaluation may occur. For example, an upper endoscopy may be performed on the patient to inspect and evaluate the patient. Namely, the patient's upper digestive tract, including the esophagus, stomach, and duodenum, may be evaluated. Similar to above, if the evaluation indicates a higher than acceptable risk, the condensed surgical treatment process will be accordingly terminated.

At a step 128, bariatric surgery may be performed on the patient. A variety of bariatric surgeries, now known or later developed, may be performed at step 128. Some exemplary types of bariatric surgeries include laparoscopic adjustable gastric band, sleeve gastrectomy, and biliopancreatic diversion. In one or more embodiments, a safe sleeve gastrectomy may be performed, such as described in U.S. Pat. No. 10,016,295, which is incorporated herein by reference.

At a step 132, post-operative procedures may occur. For instance, IV therapy, fluids or both may be administered. A consultation may also accompany the same.

At a step 136, the patient may proceed to any scheduled travel or stay arrangements scheduled at step 116. For example, the patient may travel home (or elsewhere) via scheduled flight, ground, or other transportation. Alternatively, the patient may stay at scheduled lodging arrangements, and be provided scheduled transport to the same.

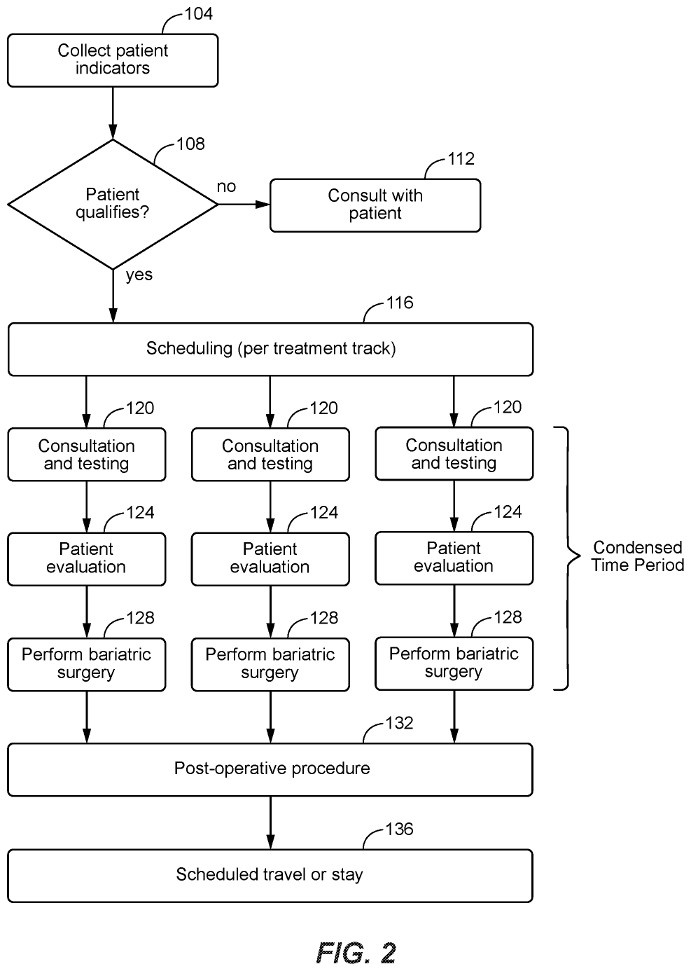

FIG. 2 is a flow diagram illustrating a condensed surgical treatment process having multiple treatment tracks. As can be seen, separate and distinct consultation and testing, patient evaluation, and bariatric surgery steps 120, 124, 128 can be scheduled for different patients. Such distinct scheduling will typically be based on the patient indicators collected at step 104. For example, one patient's indicators may trigger a desire or need for distinct or additional consultation and testing, patient evaluation, and bariatric surgery steps 120, 124, 128 relative to other patients.

FIG. 2 also illustrates an advantage of the qualification process using patient indicators at step 104 and decision step 108. As can be seen, the consultation and testing, patient evaluation, and bariatric surgery steps 120, 124, 128, even if distinct, can be scheduled within a predefined condensed time period if the patient is qualified at decision step 108.

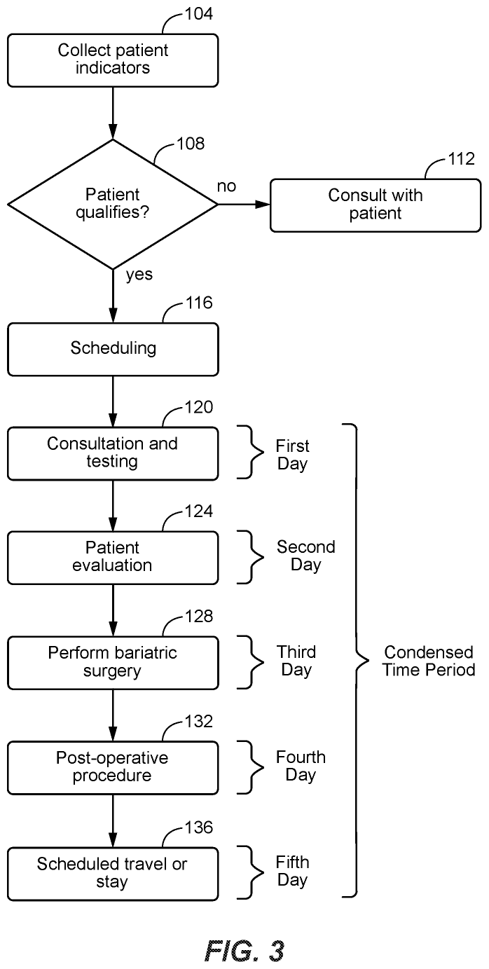

Referring to FIG. 3, it can be seen that individual steps of the condensed surgical treatment process will typically be scheduled to occur within predefined divisions of time within an overall predefined condensed time period. This allows the condensed surgical treatment process to occur within the predefined condensed time period, thereby giving the patient a well-defined timeframe so that they may plan accordingly and avoid undue interruption and disruption to their lives. In addition, as described above, the condensed surgical treatment process provides a condensed time frame relative to traditional bariatric surgery.

In the embodiment of FIG. 3, the consultation and testing, patient evaluation, bariatric surgery, post-operative procedure, and scheduled travel or stay steps 120, 124, 128, 132, 136 each occur within a division of time of a day, with the overall condensed time period being five days. It is contemplated that, in some embodiments, the divisions of time may be distinct, longer, or even shorter than that shown. This may be achieved by the qualification process of step 104 and decision step 108 and scheduling testing and evaluation of step 120 and step 124 with providers that are in-house or are otherwise capable of providing a turnaround time within a particular division of time. Typically, there will not be any intervening period of time between the division of times.

It is contemplated that a condensed time period need not include all the steps described above. For example, in one embodiment, a condensed time period may not include a post-operative procedure step 132, a scheduled travel or stay step 136, or both. In such case, the condensed time period provided to the patient will be that of the consultation and testing, patient evaluation, bariatric surgery steps 120, 124, 128. The post-operative procedure and scheduled travel or stay steps 132, 136 may still be provided but need not be scheduled to occur within a defined division of time.

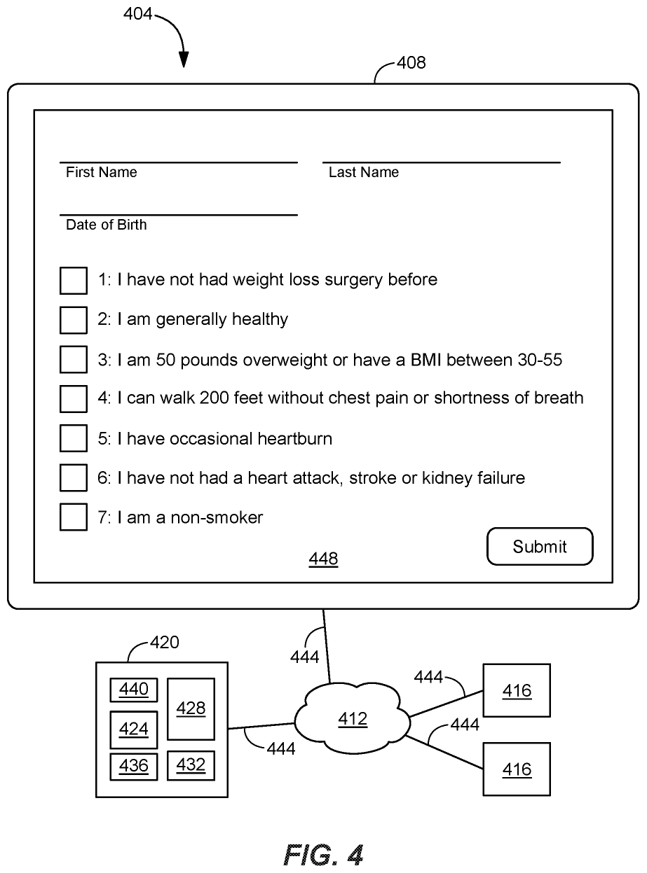

FIG. 4 is a block diagram illustrating an exemplary qualification system 404. As can be seen, the qualification system 404 may comprise one or more servers 420 in communication with one or more patient client devices 408, one or more provider client devices 416, or both. The patient and provider client devices 408, 416 may be computing devices, such as smartphones, tablets, laptop or desktop computers, terminals, or the like that are capable of receiving user input and communicating with a server 420 to allow interaction therewith. For example, a patient or provider client device 408, 416 may allow interaction with a server 420 via a web or other user interface, a database connection, or the like.

Communication may occur in various ways now known or later developed. As shown for example, communication may occur via one or more wired or wireless communication links 444 and networks 412, such as one or more WANs, LANs, the internet, or various combinations thereof. Though not illustrated, communication may also occur via a direct connection. For example, a provider client device 416 may have a direct connection to a server 420 in some embodiments.

In operation, a patient client device 408 may collect patient indicators, such as via a collection interface 448 presented on a display of the patient client device. The patient may provide and self-select individual patient indicators via an input device, such as a touch screen, mouse, keyboard, or other human interface device.

As shown in FIG. 4, a collection interface 448 may include patient indicators such as date of birth, surgical history, health status, BMI, physical capabilities/characteristics, existing conditions, medical history, or whether the patient is a smoker. The exemplary embodiment of FIG. 4 provides seven patient indicators that may be provided by engaging or disengaging their associated checkboxes. For example, engaging or checking a checkbox may affirm the patient indicator, while disengaging or unchecking the checkbox would do the opposite.

As can be seen, a collection interface 448 may also receive other patient information, such as identifying information, including patient names, social security numbers, or the like. A “Submit” button or the like may be provided to allow a patient to indicate that they have completed their input and to initiate transmission of the patient indicators to a server 420.

Individual patient indicators may involve multiple physical characteristics, which a patient may affirm or not affirm together, as shown in the following exemplary set of patient indicators.

TABLE 1

| | |
|---|---|
| 1 | I have not had weight loss surgery before |
| 2 | I am generally healthy, have occasional heartburn |
| | and have not had a heart attack, stroke or |
| | kidney failure |
| 3 | I am 50 pounds overweight or have a BMI between |
| | 30-55 |
| 4 | I can walk 200 feet without chest pain or shortness |
| | of breath and I am a non-smoker |
| 5 | I have commercial insurance coverage |
| 6 | I've researched weight loss surgery and believe |
| | it is right for me |

In one or more embodiments, the collection interface 448 or checkboxes and other input fields thereof may be generated or retrieved from a server 420 for presentation via a patient client device 408. A server 420 may store one or more collection interfaces 448 and input fields thereof on a storage device 428 in a non-transient manner, which excludes carrier waves and other signaling. For example, a server 420 may have or access one or more storage devices 428 that utilize optical, magnetic, or solid-state media to store data.

Patient indicators and other patient information may be received from patient client devices 408 via one or more communication devices 432, such as a network interface card, modem, or the like. As described above, such communication may occur via wired or wireless communication links 444, networks 412, or both. The patient indicators may be stored on a storage device 428 of the server 420 as well.

It is contemplated that a server 420 may receive patient information, including patient indicators, in other ways. For example, patient information stored in paper records may be scanned and transmitted to a server 420.

Patient indicators and other patient information can be queried or reviewed by providers via a provider client device 416 in communication with a server 420. The server 420 may retrieve the patient information from one or more storage devices 428 and communicate the same to a provider client device 416 via a communication device 432, such as those described above.

A provider client device 416 may also be used for administrative purposes. For instance, a provider client device 416 may be used to create a collection interface 448 or the input fields thereof and communicate the same to a server 420 for storage. Alternatively, it is contemplated that one or more I/O ports 440 of a server may be used to create collection interfaces 448 and their input fields directly at a server 420. For example, a provider may use a keyboard, mouse, or other human interface device in connection with an I/O port 440 to create the same.

A server 420 may also comprise one or more processors 424 that execute instructions to provide the functionality disclosed herein. Some exemplary processors 424 include microprocessors, CPUs, integrated circuits, microcontrollers, and the like. Such instructions may be stored on a storage device 428 that is part of or accessible to the server 420. Alternatively, a processor 424 may be hardwired with the instructions. One or more memory devices 436, such as RAM, may be provided for temporary random-access storage.

The instructions executable by a processor 424 may also process patient indicators for qualification purposes. For instance, the instructions may qualify patients that have a particular set of patient indicators. A notification of the same may be automatically transmitted to a provider client device 416 via a communication device 432 or be added to the patients' patient information, which may be viewed at a provider client device. In the exemplary embodiment of FIG. 4 for example, a patient may be qualified at decision step 108 if each of the seven patient indicators are affirmed by the patient checking their associated check boxes.

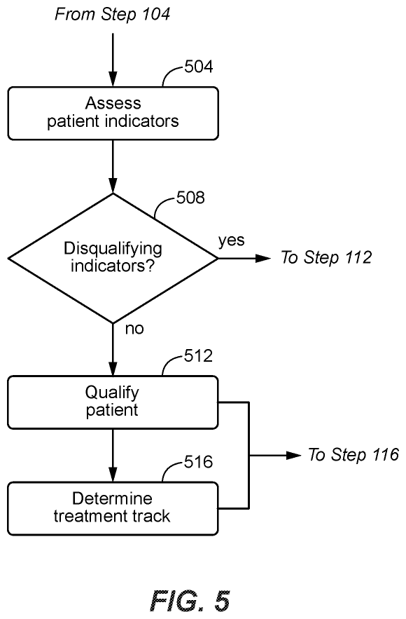

FIG. 5 is a flow diagram illustrating an exemplary qualification process that may take place at decision step 108. At a step 504, patient indicators may be assessed. This may occur in various ways. As describe above for example, in some embodiments, assessment may occur by determining whether all patient indicators have been affirmed. Alternatively, assessment may occur by determining whether the affirmed patient indicators match one or more sets of patient indicators that would allow a patient to be qualified.

At a decision step 508, if the patient has disqualifying patient indicators, the patient is disqualified and a condensed surgical treatment process may continue to step 112 for patient consultation as described above. Disqualifying patient indicators may be patient indicators that must be affirmed or must be unaffirmed which the patient does not affirm or does affirm, respectively speaking. Alternatively, or in addition, a disqualifying patient indicator may be a combination of unaffirmed or affirmed patient indicators.

If no disqualifying patient indicators are present, the patient may be qualified at a step 512. As can be seen, a condensed surgical treatment process may then proceed to step 116 for scheduling as described above. In embodiments with multiple treatment tracks, a condensed surgical treatment process may proceed to a step 516 where a patient's treatment track is selected before scheduling occurs at step 116. The selection of a treatment track will typically be based on the patient indicators.

Referring back to FIG. 4, in one or more embodiments a server 420 may store, on a storage device 428, a set of providers as well as a calendar for scheduling purposes at step 116. Typically, the set of providers only includes providers that are in-house or that are otherwise capable of providing a turnaround time within a given division of time. Instructions that provide the scheduling functionality disclosed herein may also be stored on a storage device 428 for execution by a processor 424 during operation.

After a patient is qualified at decision step 108, appointments for a patient's consultation and testing, patient evaluation, and bariatric surgery, and optionally, the patient's subsequent post-operative procedure, and scheduled travel or stay may be entered into the calendar in sequence such as shown in steps 120, step 124, step 128, step 132, and step 136 in FIG. 1. It is noted that some of the steps may be in a different sequence than that shown in FIG. 1. To illustrate, patient evaluation at step 124 could occur before testing at step 120 in some embodiments.

Typically, each step 120, 124, 128, 132, 136 will be scheduled in succession and stored in a calendar according to the desired division of time. Referring to FIG. 3 for example, each step 120, 124, 128, 132, 136 has been scheduled in sequence across a five-day condensed time period in which each step occurs within a given division of time without any intervening period of time between the divisions of time. Specifically, the division of time in the embodiment of FIG. 3 is a day.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A method for treating bariatric patients with bariatric surgery in a condensed time period comprising:
collecting a plurality of patient indicators from a patient via a collection interface;
determining if the patient qualifies for bariatric surgery in a condensed time period using the patient indicators; and
if the patient is qualified for bariatric surgery in a condensed time periods, scheduling and providing testing, patient evaluation, and bariatric surgery to the patient, each within a predefined division of time and without any intervening period of time therebetween.

2. The method of claim 1, wherein the patient indicators include whether the patient has had weight loss surgery before, whether the patient has a weight or BMI beyond a particular threshold, whether the patient can walk 200 feet without chest pain or shortness of breath, whether the patient is a non-smoker, and whether the patient has commercial insurance coverage.

3. The method of claim 1, wherein the patient qualifies for condensed surgical treatment if each of the plurality of patient indicators is affirmed.

4. The method of claim 1, wherein the patient qualifies for condensed surgical treatment if one or more predefined subsets of the plurality of patient indicators is affirmed.

5. The method of claim 1, wherein the predefined division of time is one day.

6. A method for condensed surgical treatment of bariatric patients comprising:
collecting a plurality of patient indicators from a patient via a collection interface;
determining if the patient qualifies for condensed surgical treatment using the patient indicators; and
if the patient is qualified for condensed surgical treatment, scheduling and providing testing, patient evaluation, and bariatric surgery to the patient, each within a predefined division of time.

7. The method of claim 6, wherein the patient indicators include whether the patient has had weight loss surgery before, whether the patient has a weight or BMI beyond a particular threshold, whether the patient can walk 200 feet without chest pain or shortness of breath, whether the patient is a non-smoker, and whether the patient has commercial insurance coverage.

8. The method of claim 6, wherein the patient qualifies for condensed surgical treatment if each of the plurality of patient indicators is affirmed.

9. The method of claim 6, wherein the patient qualifies for condensed surgical treatment if one or more predefined subsets of the plurality of patient indicators is affirmed.

10. The method of claim 6, wherein the predefined division of time is one day.

11. The method of claim 6, further comprising providing a post-operative procedure after the bariatric surgery.

12. The method of claim 6, further comprising scheduling travel arrangements if the patient is qualified.
